(19) United States
(12) Patent Application Publication
(10) Pub. No.: US 2025/0344989 A1
(43) Pub. Date: Nov. 13, 2025

(54) A MOBILITY SYSTEM AND A RELATED CONTROLLER, METHOD, SOFTWARE AND COMPUTER-READABLE MEDIUM

(71) Applicant: FOUNDATION FOR RESEARCH AND TECHNOLOGY-HELLAS, CRETE (GR)

(72) Inventors: VANGELIS SAKKALIS, CRETE (GR); MYRTO KRANA, CRETE (GR); CRISTINA FARMAKI, CRETE (GR); MATTHAIOS PEDIADITIS, CRETE (GR)

(73) Assignee: FOUNDATION FOR RESEARCH AND TECHNOLOGY-HELLAS, CRETE (GR)

(21) Appl. No.: 18/870,518

(22) PCT Filed: Jun. 30, 2022
(86) PCT No.: PCT/EP2022/068035

§ 371 Date: Nov. 29, 2024

(30) Foreign Application Priority Data
May 30, 2022 (GR) 20220100450

(51) Int. Cl. A61B 5/378; A61B 5/00; A61G 5/04

(52) U.S. Cl. CPC A61B 5/378; A61B 5/7264; A61G 5/04; A61G 2203/18; A61G 2203/30

(57) ABSTRACT

A mobility system and a method for controlling it. The system comprises an electric wheelchair (2); an electroencephalogram, EEG, system (3) configured to detect steady state visual evoked potentials, SSVEPs, and to record EEG data; a vision system (4) comprising a display (5) and a camera (6), and the display (5) is configured to show visual stimuli; a controller. The method comprises controlling the EEG system to record EEG data; controlling the camera to capture a digital video or image and executing an algorithm to detect an object or path; controlling the display to show first visual stimuli overlapping or pointing towards the path or object; processing the EEG data to determine if first SSVEPs are generated; and if first SSVEPs are generated, controlling the electric wheelchair to move towards the object or via said path. Also, a controller, a computer program and a computer-readable medium.

### TECHNICAL FIELD

The present disclosure concerns a mobility system which comprises an electric wheelchair. The mobility system is particularly suitable for people with reduced physical mobility, especially those suffering from a locked-in syndrome. The present disclosure further concerns a method for controlling a mobility system, a controller which can be used for implementing the method, a related software and a computer-readable medium. The controller may be a computer system or computer means, and can be used in the mobility system.

### BACKGROUND

There are known mobility systems comprising electric wheelchairs for use by persons with reduced physical mobility, and particularly those suffering from “locked in syndrome”. The locked in syndrome (LIS) is a particularly severe neurological disorder in which there is a paralysis of all voluntary muscles except for the ones that control the eye movement. For this reason, there are previously known methods in which the control of a mobility system by a person with LIS is related to eye tracking technologies. However, such previously known control methods are difficult to implement, are slow in their execution, and may cause the discomfort of the mobility system's user. These problems are solved by the present invention.

### SUMMARY OF THE INVENTION

The present invention is directed to a mobility system which is particularly suitable for use by paraplegic persons or persons with LIS. This system, as well as other related aspects of the invention, allow for an improved comfort of the user, an improved level of control by the user, and an improved functionality compared to previously known mobility systems. The present invention particularly achieves shortening the time required for navigating using the wheelchair. The present invention also allows for upgrading previously known mobility systems. Overall, the invention offers advanced functionalities as well as comfortable use of the mobility system by its user.

The present invention in a first aspect concerns a method for controlling a mobility system, the mobility system comprising: an electric wheelchair; an electroencephalogram, EEG, system configured to detect steady state visual evoked potentials, SSVEPs, and to record EEG data; a vision system comprising a display and a camera which is configured to capture a video or image of a scene, and the display is configured to show visual stimuli and to enable a view of at least a part of the scene; a controller configured to operatively communicate with and control the vision system and the electric wheelchair, and the controller is also configured to operatively communicate with the EEG system. The method comprises the steps of:
a) the controller controlling the EEG system to record EEG data;
b) the controller controlling the camera to capture the digital video or image of the scene, processing the digital video or image and executing an algorithm to detect an object or path in the scene;
c) if in step (b) the object or path is detected, the controller controls the display to show first visual stimuli within the view which is enabled by the display, the first visual stimuli overlapping or pointing towards the path or object;
d) the controller processing the EEG data to determine if first SSVEPs are generated in response to the first visual stimuli;
e) if in step (d) the controller determines that first SSVEPs are generated, the controller controlling the electric wheelchair to move towards the object or via said path.

Preferably the system's display is augmented reality, AR, glasses, and part or whole of the scene captured by the camera can be viewed (i.e. be visible) via (through) or by means of the AR glasses. Hence, the scene or part of it can be within a field of view of the AR glasses. The AR glasses may be configured to display visual stimuli within said field of view, and preferably the controller in step (c) controls the AR glasses to display the first visual stimuli within said field of view, the first visual stimuli overlapping or pointing towards the object or path. It can be understood that when the display is AR glasses, the latter may comprise transparent screens via which a user of the AR glasses can view the scene or part of it. This way the AR glasses would enable the view of at least a part of the scene. However, there are other types of AR glasses which operate in a different manner for enabling a view of a scene around the glasses. Alternatively, the display may be of a different type, e.g. be a monitor, which could enable a view of the scene by showing, i.e. displaying, the image or video which is captured with the camera.

The mobility's system controller, which may also be called computer or computer means, may generally comprise one or more computers and/or microcontrollers which are interconnected and interact for combinedly executing the method steps (a)-(e), as well as for optionally executing many of the other steps described below. Hence, the controller may comprise one or more CPUs, microcontrollers or other similar components, and may also comprise one or more electronic memories for storing information. The controller may also comprise one or more electronic interfaces for operatively communicating or being connected with other components of the mobility system. Hence, the controller may be a single computer, or may alternatively comprise different computing parts which are respectively integrated in different components of the mobility system, e.g. in the vision and the EEG systems. Similarly, the controller or a software run by the controller may preferably comprise different modules related to the different method steps. Said optional modules may have the form of corresponding software, hardware or combinations of software and hardware.

Hence, the present invention in another aspect concerns a controller for a mobility system, the controller comprising: a processor; a memory; one or more interfaces for operatively communicating with and controlling an electric wheelchair, an electroencephalogram, EEG, system, and a vision system, wherein the vision system comprises a display and a camera which is configured to capture a video or image of a scene, the display is configured to enable a view of the scene and to show visual stimuli, the EEG system is configured to detect steady state visual evoked potentials, SSVEPs, and to record EEG data; and wherein the controller further comprises an EEG control module for controlling the EEG system to record EEG data; a camera control module for controlling the camera to capture the digital video or image of the scene; an image processing module for processing the digital video or image, and for executing an algorithm to detect an object or path in the scene; a display control module for controlling the display to show first visual stimuli within the view which is enabled by the display, the first visual stimuli overlapping or pointing towards the path or object; an EEG processing module for processing the EEG data to determine if SSVEPs are generated in response to the corresponding visual stimuli; and a wheelchair control module for controlling the electric wheelchair to move towards the object or via the path detected with the image processing module.

Hence, the present invention in another aspect concerns a mobility system which comprises: an electric wheelchair; an electroencephalogram, EEG, system configured to detect steady state visual evoked potentials, SSVEPs, and to record EEG data; a vision system comprising a display and a camera which is configured to capture a video or image of a scene, and the display is configured to show visual stimuli and to enable a view of at least a part of the scene; a controller configured to operatively communicate with and control the vision system and the electric wheelchair, and the controller is also configured to operatively communicate with the EEG system; wherein the controller is adapted to execute the steps of the method of the first aspect of the invention.

The present invention in another aspect concerns a computer program comprising instructions to cause a controller in a mobility system which is according to the invention, to execute the steps of a method which is according to the invention. Similarly, the present invention in another aspect concerns a computer-readable medium having stored thereon the aforementioned computer program.

With respect to the method according to the invention, step (b) is particularly important because it enables, i.e. offers, an automatic identification of an object or path. In turn, said automatic identification allows, via steps (a), (c) and (d), for the direct selection by the user of said object or path. Said direct selection can in turn enable, via step (e), an automatic movement towards the selected object or path. Therefore, steps (a)-(e) combinedly allow for the user avoiding having to “manually” or stepwise navigate towards/via said object/path, wherein “manually” means breaking down and executing the navigation into segments, e.g. first go straight, then left, then straight again, then right etc. Navigating manually, i.e. in segments, can be a particularly challenging, slow and tiresome task for a person with LIS, because the selection of each different segment generally requires an appreciable amount of time and effort by the user. In contrast, with the present invention, the algorithm implemented in step (b) allows the user to perform only one selection, i.e. select once the object or path of interest, even if moving towards the selected object or path entails executing a multi-segment trip. After the user has done said single selection, the method allows navigation without the user having to provide additional selections (inputs). Advantageously, this offers a control method which is appreciably faster and less tiresome compared to previously known methods. Moreover, using the display to present the visual stimuli, and having said stimuli overlapping or pointing towards the object or path of interest, provides a control method which is immersive, and which the user can easily comprehend and follow.

In an embodiment of the invention, executing the algorithm in step (b) comprises using a neural network, preferably a convolutional neural network, for detecting the object or path in the scene. Advantageously, the use of a neural network for the detection of the object or path is easy to implement, at least due to the significant advancements which have been recently made in the field of neural networks. Moreover, the use of neural networks can also advantageously allow the training or feedback of the neural network by past actions of the mobility system's users, so that the accuracy and speed of the method can progressively be improved.

The method according to the invention can be combined with steps related to a manual navigation wherein the user gives distinct commands for executing a series of forward or sideway motions for doing respective segments of a trip along a path or towards certain destination. Said commands may be given by means of the user starring at different or “second” visual stimuli compared to the visual stimuli used for the selection of a complete path or object. The second visual stimuli would be specifically related to the commands for forward, turning or sideways motion, and can be projected on the display. The EEG system may detect SSVEPs caused by the user starring at said second visual stimuli. The EEG data recorded by the EEG system may be processed by the controller so that the latter can control the wheelchair to do distinct forward, turning and/or sideways movements. It is noted that it is also contemplated the option of including commands and corresponding visual stimuli related to a backward motion of the wheelchair, however, for safety reasons this is not a preferable option because it may be unsafe if the wheelchair moves backwards. According to the above, a preferred embodiment of the method comprises the aforementioned steps (a)-(e), and further comprises the steps of:
f) the controller controlling the display to show second visual stimuli;
g) the controller processing the EEG data to determine if second SSVEPs are generated in response to the second visual stimuli;
h) if in step (g) determining that the second SSVEPs are generated, the controller controlling the electric wheelchair to move forward, left or right.

Preferably the first and second visual stimuli are graphics which flicker at the same or different frequencies with respect to each other. Moreover, preferably the second visual stimuli comprise three flickering checkerboards each of which is capable of causing respective second SSVEPS for causing a respective one of the forward, left or right movement in step (h). Hence, the user by looking at each one of said three targets may command the system to move forward, left or right.

The first and/or the second visual frequency preferably are graphics which flicker on a screen or screens of the display. The frequency at which said graphics may flicker can determine the frequency of the corresponding SSVEPs. Hence, in an embodiment which comprises the aforementioned optional steps (f)-(h), for distinguishing between different SSVEPs which correspond to different types of visual stimuli, the first and second visual stimuli flicker at different frequencies with respect to each other. However, there is also contemplated the possibility that steps (a)-(e) are executed in different moments compared to steps (f)-(h), in which case, the first and second visual stimuli would not be simultaneously displayed on the display, and hence, would not have to flicker at different frequencies. Therefore, optionally the first and second visual stimuli may flicker at the same frequencies with respect to each other. Preferably, the visual stimuli, e.g. the first and/or the optional second visual stimuli, are flickering graphics, more preferably flickering targets or checkerboards. Flickering checkerboards can cause strong SSVEPs which can be detected with the EEG system.

In the mobility system according to the invention, the field of view of the camera may be broader than the part of the scene being displayed by the display. Likewise, if the display via which a user views the scene is AR glasses, the scene captured by said camera may be larger than what the user can see though the AR glasses. For example, when said user has limited mobility or field of vision, and he cannot turn his head for looking at different things, his field of vision may be smaller compared to the field of view of the camera. In these situations where there are objects or paths of interest which are beyond what the user sees through or by the display, the system may advantageously be configured so that the corresponding projected visual stimuli point towards said objects. Hence, in an embodiment of the method according to the invention, the display enables viewing a part of the scene, the object or path in step (b) is outside the part of the scene, and the first visual stimuli in step (c) comprise arrows pointing towards the object or path.

According to the information provided above, it may be understood that steps (c)-(e) may be part of a first, rather advanced, operation mode of the mobility system, said first mode offering to the user an automatic fast navigation, whereas steps (f)-(h) may be part of a second operation mode of the mobility system, said second mode offering a “manual” navigation scheme which relies on giving a series of different commands for going forward, left and/or right. Hence, the method may comprise additional steps for the user choosing between the two modes, or for the user confirming whether to proceed with the advanced mode. Hence, a preferred embodiment which is according to the first aspect of the invention, further comprises the following steps after step (b) and before step (c):
b1) if in step (b) the object or path is detected, the controller controlling the display or a speaker of the system to indicate the detection of the object or path;
b2) the controller controlling the screen to display confirmation visual stimuli;
b3) the controller processing the EEG data to determine if confirmation SSVEPs are generated in response to the confirmation visual stimuli;
b4) if in step (b3) it is determined that the confirmation SSVEPs are generated in response to the confirmation visual stimuli, then the controller proceeds to execute step (c).

The speaker mentioned in step (b1) is an optional component of the mobility of the system, and can be used for announcing, thereby indicating, the detection of the object. Said announcement may have the form of one or more words and/or sounds. Alternatively, in step (b1) the display can be controlled for indicating therein that the object or path is detected. The respective indication in the display may have the form of text, graphics, or combinations thereof. The confirmation visual stimuli in step (b2) may have the form of blinking graphics, e.g. graphics which flicker at a specific frequency and can cause the generation of the confirmation SSVEPs defined in step (b3). If in step (b3) it is detected that said confirmation SSVEPs are generated, which means that the mobility system's user has looked at the confirmation visual stimuli for confirming that the user wishes to proceed to step (c), then indeed the controller proceeds to execute step (c). If the confirmation SSVEPS are not detected, then the controller may control the camera to continue capturing a new image or video.

More generally, the controller may preferably be configured to be triggered for not executing step (c), and/or to be triggered for executing step (c). A preferred route via which the controller may be triggered to execute step (c), are the aforementioned optional steps (b1)-(b4). In some embodiments of a method according to the invention, the mobility system comprises a joystick or a button connected to the controller, and via said joystick or button the user may manually instruct the controller to avoid executing step (c) and the advanced navigation mode of steps (c)-(e). An alternative way for the user triggering the controller to avoid the advanced navigation mode, is that the user looks away from the display for a period time, for thereby causing a respective change or feature in the EEG data. This change may have the form of an interruption in the SSVEP generation, and may be detectable by the controller which processes the EEG data. According to the above, an embodiment of a method according to the invention, further comprises the controller determining if a condition is met for executing step (c), the condition being that the controller is not triggered to not execute step (c), and/or that the controller is triggered, preferably by means of executing steps (b1)-(b4), to execute step (c).

An embodiment of the mobility system according to the present invention can advantageously also enable controlling objects which the wheelchair's user may wish to control during the use of the wheelchair. In the latter embodiment, the mobility system further comprises a controllable object which comprises an electronic part which is operatively connectable, and can hence communicate, with the mobility system's controller for receiving from the latter control signals. These control signals can trigger an operation or function of the object. The object may for example be a television which upon receiving a control signal from the controller may switch on or off, or may change channel. In another example, the object may be a door or window having an electronically controlled actuator which is configured for opening the door or window upon receiving a respective signal from the controller. An object may be identified during the operation of the mobility system via a variety of alternative ways, e.g. via the object communicating to the controller an object identification (ID) name or number, or via the controller automatically identifying the object by means of the algorithm of step (b) of the method, or indeed by means of a different algorithm run by the controller as part of the step (b). The object's identity given by the aforementioned identification of the object, can in turn allow the controller to determine if a condition is met for the controller being able to move on to further steps for controlling the object. Said further condition may be that the object according to its identification (ID) is indeed a controllable object, and/or that the user allows—or does not object—the execution of further steps for the control of the object. In particular, the controller can check if the object's ID belongs to a respective list of IDs of controllable objects. If this condition is met, the controller may proceed with controlling the display to show control visual stimuli which are capable of causing the generation of control SSVEPS i.e. SSVEPs which are generated when the user looks at the control visual stimuli in the display. Preferably there are more than one functionality which the object can perform upon receiving respective commands from the controller, and for executing each of said commands, the user has to look at a respective control visual stimuli shown in the screen, e.g. if the controllable object is a door one visual stimuli may be for opening the door, and another control stimuli may be for locking the door. Hence, the controller may preferably control the display to show a plethora of control visual stimuli related to different respective commands for controlling the object. In addition, the controller may process the EEG data to determine if control SSVEPs are generated in response to the control visual stimuli. If the controller determines that the control SSVEPs are generated, then the controller may generate a command signal for controlling the object. If the object is not a controllable object, or if no control SSVEPs are detected, or if the user otherwise triggers/instructs the controller to not control the object, the controller may continue with other method steps, such any of the steps (a)-(h) mentioned further above. Hence, in an embodiment of the method according to the invention, step (b) comprises detecting an object, and the method further comprises the steps of: the controller identifying the object; the controller determining if according to the object's identity, a further condition is met; If the further condition is met, the controller controlling the display to show control visual stimuli, preferably a plethora of control visual stimuli related to respective commands for controlling the object; the controller processing the EEG data to determine if control SSVEPs are generated in response to the control visual stimuli; if the controller determines that the control SSVEPs are generated, the controller generating a command signal for controlling the object. In an embodiment which is similar to the previous one, controlling the object is done using the KNX standard communication protocol for smart home and building automation.

### BRIEF DESCRIPTION OF DRAWINGS

FIG. 1 is a block diagram of an embodiment of a mobility system according to the invention.

FIG. 2 is a block diagram showing the components of another embodiment of a mobility system according to the invention.

FIG. 3 is a block diagram showing the components of an embodiment of a controller according to the invention.

FIG. 4 is a flow diagram of an embodiment of a method according to the invention.

FIG. 5A is a flow diagram of an embodiment of a method according to the invention.

FIG. 5B is a flow diagram of an embodiment of a method according to the invention.

FIG. 6A is a view of a scene captured by the camera and displayed by the display.

FIG. 6B shows the view of FIG. 6A with displayed first visual stimuli overlapping an object found in the view.

FIG. 6C shows the view of FIG. 6A with displayed second visual stimuli of a navigation scheme.

### DETAILED DESCRIPTION OF EMBODIMENTS

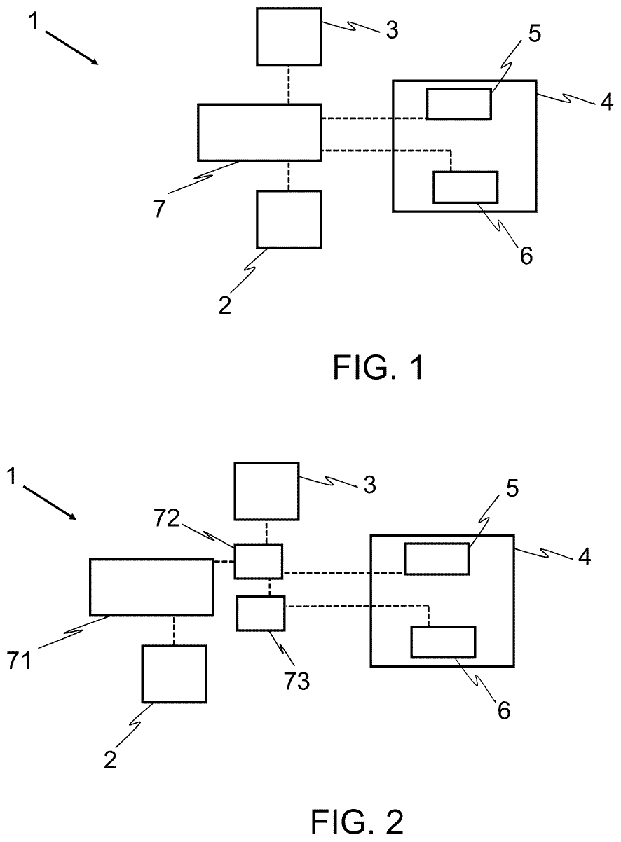

Referring to FIG. 1 which shows the main components of a preferred embodiment of a mobility system according to the invention, said mobility system comprises the following: an electric wheelchair 2; an electroencephalogram, EEG, system 3 which is configured to detect steady state visual evoked potentials, SSVEPs, and to record EEG data; a vision system 4 comprising a display 5 and a camera 6 which is configured to capture a video or image of a scene, and the display 5 is configured to show visual stimuli and to enable a view of at least a part of the scene; a controller 7 configured to operatively communicate with and control the vision system and the electric wheelchair 2, and the controller 7 is also configured to operatively communicate with the EEG system 3. The particular vision system 4 of the embodiment of FIG. 1, is AR glasses which integrate the camera 6 and the display 7, said display being two screens on which graphics can be displayed overlapping with the field of view of the user who can see through the glasses. Alternatively, the AR glasses may be of a different type, or the display may be a normal display such as a computer monitor or a television. The dashed lines in FIG. 1-3 indicate connections between the shown components. These connections may be wired, wireless or combinations thereof. Referring also to the preferred embodiment of FIG. 1, the controller specifically uses a convolutional neural network for detecting the object or path of interest. In an embodiment, the controller can also identify said object or path of interest by means of a neural network. The latter preferably is a convolutional neural network.

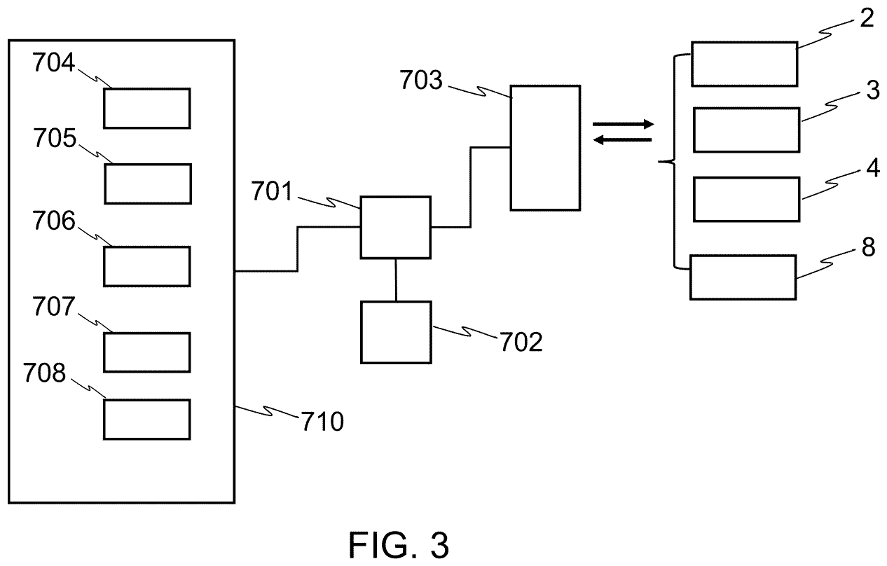

Referring to FIG. 2, the controller of the respective mobility system comprises: a wheelchair control module 71 for controlling the wheelchair; an EEG and display module 72 for processing the EEG data, as well as for controlling the display to show visual stimuli which can provoke the generation of SSVEPs to the system's user; and an image processing module 73 for receiving and processing images and/or videos captured with the camera. The aforementioned EEG and display module 72 combined with the EEG system 3 can be considered as forming or being parts of a brain-computer interface (BCI) system which effectively allows for using the user's brain activity for controlling the mobility system. The display may also be considered as part of the BCI system. Said EEG and display module 72 may comprise submodules, such as for example a graphics generation submodule which generates or selects graphics to be displayed as visual stimuli using the display, and an EEG data processing and analysis submodule. The modules and submodules of the system may have the form of hardware, software or combinations thereof.

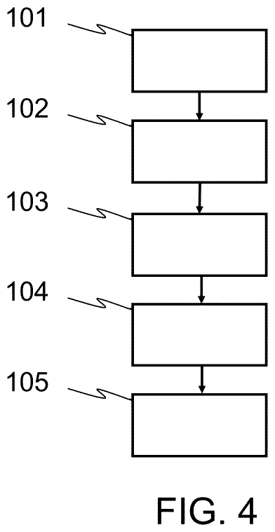

Referring to FIG. 3 which concerns an embodiment of a controller according to the invention, said controller comprises: a processor 701; a memory 702; one or more interfaces 703 for operatively communicating and controlling an electric wheelchair, an electroencephalogram, EEG, system, and a vision system, wherein the vision system comprises a display and a camera which is configured to capture a video or image of a scene, the display is configured to show visual stimuli, the EEG system is configured to detect steady state visual evoked potentials, SSVEPs, and to record EEG data; an EEG control module 704 for controlling the EEG system to record EEG data; a camera control module 705 for controlling the camera to capture the digital video or image of the scene; an image processing module 706 for processing the digital video or image, and executing an algorithm to detect an object or path in the scene; a display control module for controlling the display to show first visual stimuli within the view which is enabled by the display, the first visual stimuli overlapping or pointing towards the path or object; an EEG processing module 707 for processing the EEG data to determine if SSVEPs are generated in response to the corresponding visual stimuli; an electric wheelchair control module 708 for controlling the electric wheelchair to move towards the object, or via the path, which is detected with the image processing module. Modules 704-708 form an embodiment of a computer program which comprises instructions to cause the controller shown in FIG. 3, to execute the steps of the method according to a first aspect of the invention. The aforementioned modules 704-708 are in a computer-readable medium 710 having stored thereon the computer program. A computer readable medium which is according to the invention, may preferably be part of the controller.

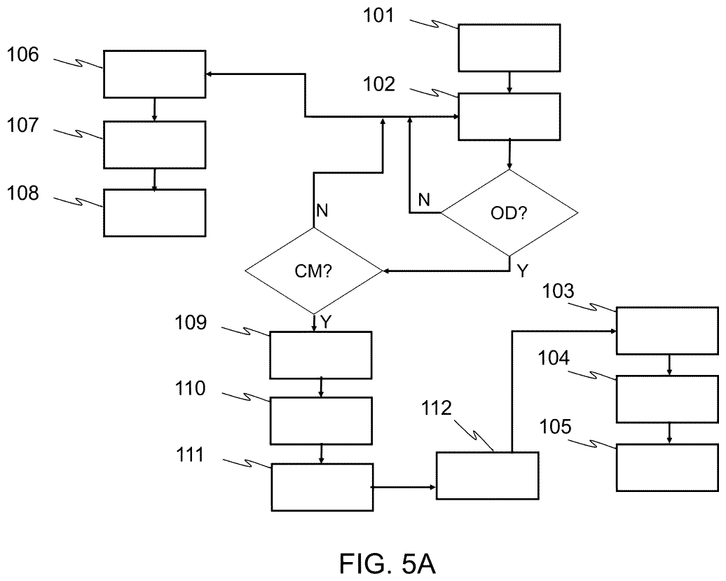

The method of the embodiment shown in FIG. 4 comprises:
Step 101 wherein the controller controls the EEG system so that the latter records EEG data.
Step 102 wherein the controller controls the camera so that the latter captures the digital video or image of the scene. Also, in step 102 the controller processes the digital video or image, and executes an algorithm to detect an object or path in the scene.
Step 103 wherein, if in step 102 the object or path is detected, the controller controls the display to show first visual stimuli within the view which is enabled by the display, the first visual stimuli overlapping or pointing towards the path or object;
Step 104 wherein the controller processes the EEG data to determine if first SSVEPs are generated in response to the first visual stimuli;
Step 105 wherein if in step 104 the controller determines that first SSVEPs are generated, the controller controls the electric wheelchair to move towards the object or via said path.

It is noted that optionally and preferably in step 102 multiple objects can be detected at the same time by executing the algorithm, or by executing a plurality of algorithms. Likewise, an alternative preferred embodiment comprises the aforementioned steps 101-105 but with the following modification: in step 102 the algorithm is executed to detect an object, and step 105 further comprises that before the controller controls the electric wheelchair, the controller or a module (e.g. a software module) of the controller plans a path towards the object. Hence, preferably in step 102 there is detected an object and not a path. Likewise, preferably step 105 further comprises planning a path towards the object detected. In another embodiment, in step 102 there is detected an object, and the method further comprises planning a path towards the object. Said optional path planning may involve the use of one or more LIDAR sensors and/or a software module configured for executing simultaneous localization and mapping (SLAM) and path planning methods as described further below.

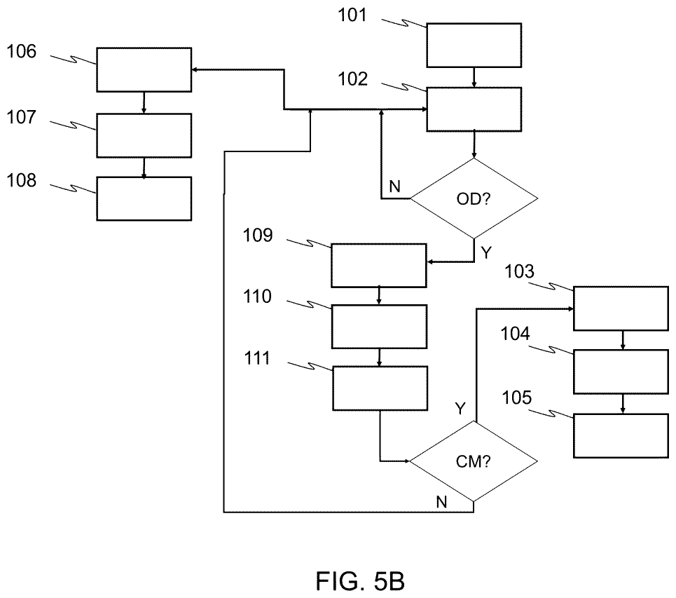

Referring to FIG. 5A, the respective embodiment further comprises the steps of:
step 106 wherein the controller controls the display to show second visual stimuli;
step 107 wherein the controller processes the EEG data to determine if second SSVEPs are generated in response to the second visual stimuli;
step 108, wherein if in step 107 determining that the second SSVEPs are generated, the controller controls the electric wheelchair to move forward, left or right.

Also, in the embodiment shown in FIG. 5A, if the object or path is not detected, i.e. if the answer to the shown logical question “OD?” (object/path detected?) is NO (“N”), then step 102—particularly the processing of the image or video—is repeated or continued, or alternatively, the controller may proceed with steps 106-108. However, if the answer to the aforementioned logical question is YES (Y), then the controller may proceed to determining if a further condition is met, i.e. whether the answer to the logical question “CM?” (Condition met?) is YES (Y) or NO (N). In a non-limiting example of a method which is similar to the one shown in FIG. 5A, the mobility system comprises an optional controllable switch, either manual or via gesture, and with said switch the user can choose whether to use an “automatic navigation” mode or not, and in said example, the condition related to the aforementioned logical question “CM?” is met if the user has selected via the switch said “automatic navigation” mode. If the answer is NO then the controller may continue with steps 106-107, or may go back to step 102, or may switch to the navigation done with steps 106-108. However, if the answer to the “CM?” question is YES then the controller may continue with steps 109-112 which are the following:
in step 109, if in step 102 the object or path is detected, the controller controls the display or a speaker of the system to indicate the detection of the object or path;
in step 110, the controller controls the screen to display confirmation visual stimuli;
in step 111, the controller processes the EEG data to determine if confirmation SSVEPs are generated in response to the confirmation visual stimuli;
in 112, if in step 111 it is determined that the confirmation SSVEPs are generated in response to the confirmation visual stimuli, then the controller proceeds to execute step 103. If in step 111 it is determined that confirmation SSVEPs are not generated, then the controller may preferably proceed to step 106, or alternatively proceed to step 102 or another step of the method.

It is noted that in a preferred embodiment which comprises the aforementioned steps 101-105 and 106-108, step 102 is run in parallel with steps 106-108, i.e. 102 is done concurrently to 106-108. In particular, preferably step 102 for object detection is executed continuously e.g. by running continuously a corresponding software module which implements step 102.

FIG. 5B shows a preferred embodiment which comprises steps 109-111 which are executed to determine if a condition is met (“CM?”). If in step 111 it is determined that the confirmation SSVEPs are generated in response to the confirmation visual stimuli, then the answer to “CM?” may be determined as being YES.

In an embodiment, the controller determines if a condition is met for executing step 103, the condition being that the controller is not triggered to not execute step 103. One of the possible ways via which the controller may be triggered to not execute step 103, is that the mobility system comprises a device, such as key or joystick, via which the user may provide an input which may trigger the controller to avoid the advanced navigation which is related to the aforementioned steps 103-105. In an alternative way, the controller may be configured to determine that the condition is not met when there are certain patterns/features in the EEG data, or when there is an absence of certain features or patterns in the EEG data. Said features or absence of features from the EEG data may be caused when the user intentionally looks away from the display or parts/graphics therein.

In an embodiment, the controller determines if a condition is met for executing step 103, the condition being that the controller is triggered to execute step 103. One preferable way for triggering the controller to execute step 103, is by means of executing the aforementioned steps 109-112.

In a non-limiting example of the method, said example described below, the vision system comprises AR glasses, the mobility system comprises a BCI system, and the users navigate the wheelchair through an AR SSVEP-based BCI system. The system can also recognize various objects, such as doors, and automatically guide the users in the direction of the identified objects, if they desire. With the use of AR glasses, the users can see the visual stimuli which are stimulation targets (4 flickering checkerboards/targets on a square lay-out, each checkerboard represents a direction command e.g FORWARD, BACKWARD, RIGHT, LEFT) projected on the screen while being aware of the surrounding environment. The built-in camera of the glasses records the environment whereas an object detection algorithm, specifically a YOLO-vs3 algorithm which employs a convolutional neural network, identifies objects in the environment. It is possible to use a different algorithm instead of the YOLO-vs3. If an object is being detected, the system asks from the user via voice message to confirm the object detection mode or to ignore it. If the user chooses the object detection mode, flickering checkerboards are projected on the identified objects and the user has to gaze on the flickering checkerboard of the desired object in order to automatically be navigated in that direction. Otherwise, the user continues the wheelchair's navigation using the “manual” navigation scheme which relies on the aforementioned direction commands (FORWARD, BACKWARD, RIGHT, LEFT).

Some details of said example are the following:
For the object detection algorithm, a pretrained model of YOLO3 is used.
Unity is used for the BCI layout
In the Navigation scheme, if the voice message (from Python) informs that there are identified objects in the environment, the user confirms the object detection mode pressing a “SPACE” key or by performing a gesture that corresponds to the “SPACE” key. Otherwise, the user ignores the message.
For projecting targets on identified objects: when “SPACE” key is pressed, targets are projected on the identified objects. Because the field of view (FV) of the AR glasses is usually smaller than the video resolution, if an object is in the video but not in the FV of glasses, arrows appear and show where to find the identified object.
The user can be released from the automatic navigation mode by pressing a “TAB” key.
The total system implementation is in Python.
Communication between Python and Unity is achieved via sockets, specifically Async sockets for Unity, in order the targets to flicker in the correct frequencies, and Sync sockets for Python.
The AR camera has 640×480 video resolution.

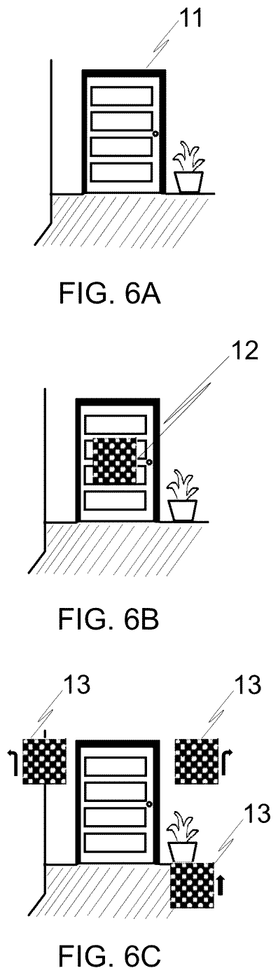

FIG. 6A shows a view of a scene captured by the camera of an embodiment, wherein within the scene one can notice a door 11, and a pot with a plant next to the door. A camera of the system may capture a view of the scene of FIG. 6A. The door may be detected by an algorithm, e.g. by an appropriately trained neural network, used by the system. The detection of the door may enable the display of the same view with a first visual stimuli 12 which has form of a flickering checkerboard overlapping the door, as shown in FIG. 6B. The user by looking at the first visual stimuli 12, may enable the system to automatically move towards the door. If the algorithm does not detect the door, or if the user opts for a navigation mode/scene which involves other steps, such as for example the aforementioned steps 106-108, then the same scene may be displayed with second visual stimuli 13, as shown in FIG. 6C. As shown in FIG. 6C the second visual stimuli 13 also have the form of checkerboards which flicker, and correspond to instruction for moving forwards or turning as indicated by the thick arrows shown next to the checkerboards.

A mobility system according to the invention preferably and optionally may further comprise LIDAR sensors and a software module configured for executing simultaneous localization and mapping (SLAM) and path planning methods. However, there are contemplated other alternative possible configurations of the system, for example one where the mobility system comprises a proximity optical sensor which is not LIDAR.

While the foregoing is directed to embodiments of the present invention, other and further embodiments of the invention may be devised without departing from the basic scope thereof. For example, other aspects may be implemented in hardware or software or in a combination of hardware and software. Additionally, the software programs included as part of the invention may be embodied in a computer program product that includes a computer useable medium, for example, a readable memory device, such as a hard drive device, a flash memory device, a CD ROM, a DVD/ROM, or a computer diskette, having computer readable program code segments stored thereon. The computer readable medium can also include a communications link, either optical, wired, or wireless, having program code segments carried thereon as digital or analog signals.

1. A method for controlling a mobility system (1), the mobility system comprising
an electric wheelchair (2);
an electroencephalogram, EEG, system (3) configured to detect steady state visual evoked potentials, SSVEPs, and to record EEG data;
a vision system (4) comprising a display (5) and a camera (6) which is configured to capture a video or image of a scene, and the display (5) is configured to show visual stimuli and to enable a view of at least a part of the scene;
a controller (7) configured to operatively communicate with and control the vision system and the electric wheelchair (2), and the controller (7) is also configured to operatively communicate with the EEG system (3);
wherein the method comprises the steps of:
a) the controller controlling the EEG system to record EEG data;
b) the controller controlling the camera to capture the digital video or image of the scene, processing the digital video or image, and executing an algorithm to detect an object or path in the scene;
c) if in step (b) the object or path is detected, the controller controlling the display to show first visual stimuli within the view which is enabled by the display, the first visual stimuli overlapping or pointing towards the path or object;
d) the controller processing the EEG data to determine if first SSVEPs are generated in response to the first visual stimuli;
e) if in step (d) the controller determines that first SSVEPs are generated, the controller controlling the electric wheelchair to move towards the object or via said path.

2. A method according to claim 1, wherein executing the algorithm in step (b) comprises using a neural network, preferably a convolutional neural network, for detecting the object or path in the scene.

3. A method according to claim 1, wherein the method further comprises the steps of:
f) the controller controlling the display to show second visual stimuli;
g) the controller processing the EEG data to determine if second SSVEPs are generated in response to the second visual stimuli;
h) if in step (g) determining that the second SSVEPs are generated, the controller controlling the electric wheelchair to move forward, left or right.

4. A method according to claim 3, wherein the first and second visual stimuli are graphics which flicker at the same or different frequencies with respect to each other.

5. A method according to claim 3, wherein the second visual stimuli comprise three flickering checkerboards each of which capable of causing respective second SSVEPS for causing a respective one of the forward, left or right movement in step (h).

6. A method according to claim 1, wherein the visual stimuli comprise flickering graphics, preferably flickering targets or checkerboards.

7. A method according to claim 1, wherein the display enables viewing a part of the scene, the object or path in step (a) is outside the part of the scene, and the first visual stimuli in step (b) comprise arrows pointing towards the object or path.

8. A method according to claim 1, wherein the display is augmented reality, AR, glasses, or is a monitor.

9. A method according to claim 1, wherein the method further comprises the following steps after step (b) and before step (c):
b1) if in step (b) the object or path is detected, the controller controlling the display or a speaker of the system to indicate the detection of the object or path;
b2) the controller controlling the screen to display confirmation visual stimuli;
b3) the controller processing the EEG data to determine if confirmation SSVEPs are generated in response to the confirmation visual stimuli;
b4) if in step (b3) determining that the confirmation SSVEPs are generated in response to the confirmation visual stimuli, then the controller proceeds to execute step (c).

10. A method according to claim 9, wherein the controller determining if a condition is met for executing step (c), the condition being that the controller is not triggered to not execute step (c), and/or that the controller is triggered, preferably by means of executing steps (b1)-(b4), to execute step (c).

11. A method according to claim 1, wherein in step (b) detecting an object, and the method further comprises:
the controller identifying the object;
the controller determining if according to the object's identity, a further condition is met;
If the further condition is met, the controller controlling the display to show control visual stimuli, preferably a plethora of control visual stimuli related to respective commands for controlling the object;
the controller processing the EEG data to determine if control SSVEPs are generated in response to the control visual stimuli;
if the controller determines that the control SSVEPs are generated, the controller generating a command signal for controlling the object.

12. A mobility system comprising:
an electric wheelchair;
an electroencephalogram, EEG, system configured to detect steady state visual evoked potentials, SSVEPs, and to record EEG data;
a vision system comprising a display and a camera which is configured to capture a video or image of a scene, and the display is configured to show visual stimuli and to enable a view of at least a part of the scene;
a controller configured to operatively communicate with and control the vision system and the electric wheelchair, and the controller is also configured to operatively communicate with the EEG system;
wherein the controller is adapted to execute the steps of the method of claim 1.

13. A controller for a mobility system, the controller comprising:
a processor (701);
a memory (702);
one or more interfaces (703) for operatively communicating and controlling an electric wheelchair, an electroencephalogram, EEG, system, and a vision system, wherein the vision comprises a display and a camera which is configured to capture a video or image of a scene, the display is configured to show visual stimuli, the EEG system is configured to detect steady state visual evoked potentials, SSVEPs, and to record EEG data;
and wherein the controller further comprises
an EEG control module (704) for controlling the EEG system to record EEG data;
a camera control module (705) for controlling the camera to capture the digital video or image of the scene;
an image processing module (706) for processing the digital video or image, and executing an algorithm to detect an object or path in the scene;
a display control module for controlling the display to show first visual stimuli within the view which is enabled by the display, the first visual stimuli overlapping or pointing towards the path or object;
an EEG processing module (707) for processing the EEG data to determine if SSVEPs are generated in response to the corresponding visual stimuli;
an electric wheelchair control module (708) for controlling the electric wheelchair to move towards the object, or via the path, which is detected with the image processing module.

14. A controller according to claim 13, wherein the image processing module comprises a neural network, preferably a convolutional neural network.

15. (canceled)

16. (canceled)
